(19) United States
(12) Patent Application Publication
(10) Pub. No.: US 2008/0053434 A1
(43) Pub. Date: Mar. 6, 2008

(54) Mouth Prop

(75) Inventors: Paul Wightman, Oxford (GB); Andrew Atkinson, Oxford (GB); Ben Wightman, Oxford (GB); Jane Shepherd, Middlesex (GB)

Correspondence Address: YOUNG & THOMPSON, 745 SOUTH 23RD STREET, 2ND FLOOR, ARLINGTON, VA, 22202, US

(73) Assignee: Owen Greenings & Mumford Limited, Oxford (GB)

(21) Appl. No.: 11/663,210

(22) PCT Filed: Sep. 20, 2005
(86) PCT No.: PCT/GB05/03611

§ 371 Date: Sep. 12, 2007

(30) Foreign Application Priority Data
Sep. 20, 2004 (GB) 0420803.9
Sep. 1, 2005 (GB) 0517739.9

(51) Int. Cl. A61B 1/32; A61C 17/10

(52) U.S. Cl. 128/200.26; 128/207.14; 433/93; 600/238

(57) ABSTRACT

A mouth prop for insertion in use between the opposed tooth or gum regions of the patient's mandible or maxilla to space the patient's incisors thereapart, the mouth prop having a grippable portion which a clinician can use for assisting in the location and removal of the mouth prop and can be supplied with elements for delivering and/or sampling gases to or in the mouth and/or nasal region of a patient.

This invention relates to a mouth prop and in particular, but not exclusively, to mouth props (otherwise known as a bite block) for use in anaesthesia and sedation.

There are various clinical procedures in which the patient has a tube inserted into the mouth and down the throat. For example, in general anaesthesia for major surgery, the majority of patients have an airway tube inserted into the mouth and down the throat as part of the airway management. This tube may be generally of two types, namely an oral endotracheal tube (ETT) or a laryngeal mask airway (LMA). As well as securing the patient's airway and as a means of introducing the anaesthetic, the above tubes also allow monitoring of end tidal CO₂. This is done by connecting a piece of equipment into the tube that can sample the exhaled air and report the CO₂ level. Anaesthetists use this to monitor the patient's heath while unconscious.

During other clinical procedures, especially where local anaesthetics are used, patients are merely sedated rather than rendered unconscious. However, even with these procedures it is desirable that clinicians' have the ability to monitor a patient's wellbeing by sampling exhaled carbon dioxide.

One type of procedure where patients are generally sedated is gastroscopy. An airway tube is not inserted down the throat in these cases as the patient is not so deeply sedated that the airway needs to be maintained for them. However in this instance the endoscope hose is passed through the patient's mouth and down the back of their throat.

Similarly, where transoesophageal echocardiography is undertaken, an instrument is inserted into the oesophagus in order to measure the function of the heart. This procedure therefore also involves the passage of an instrument through the patient's mouth and down the back of the throat.

In all of the above procedures, there is a need to prevent damage to the patient's dentition and/or occlusion or damage to the tube or hose or instrument by the patient closing their incisors onto the tube or hose or instrument. This is particularly so when coming round from a general anaesthetic because patients quite often go into spasm and clamp hard down with their teeth.

There are various methods to try and alleviate this problem. A gum guard can be used to cover the upper incisors but these tend to be a poor fit and do not prevent biting. A gum guard also clutters the front of the mouth and, if accidentally dislodged, may drop into the back of the throat. Use is often made of a green swab bite block made by hand in the operating theatre using hospital gauze wrapped in sticking plaster and then inserted down the side of the mouth between the back molars on one side. However this is time consuming and the end product is not dimensionally stable. There are also dental mouth props but these tend to be designed to hold the mouth fully open to allow access for the dentist rather than simply to ensure the spacing between the incisors is a little more than the width of the tube (typically 16-22 mm).

GB 2,366,735 discloses a bite block comprising a portion designed to fit between the opposed tooth or gum portions of a patient's maxilla and mandible. However, this proposal does not provide secure gripping regions to allow precise and secure location of the bite block in use, and may become dislodged.

There is therefore a need for a mouth prop designed to hold the patient's incisors at a required spacing whilst being atraumatic to the gums and which allows the device suitably to be grasped to allow secure and precise location without cluttering the mouth of the patient.

Accordingly in one aspect this invention provides a mouth prop comprising a body portion including a stem at opposite ends of which are provided a tooth propping portion and a manually grippable portion, said tooth propping portion being formed such that it may be inserted in use between the opposed tooth or gum regions of a patient's mandible or maxilla to space the patient's incisors, the manually grippable portion having transversely arranged cross guard portions around which in use a clinician may hook their first and second fingers, for assisting location and removal of the mouth prop.

Preferably, the cross guard portions are sized to prevent swallowing of the mouth prop.

The manually grippable portion preferably further includes a thumb grip portion disposed to allow a clinician in use to hold the prop securely by hooking their first and second fingers around the cross guard portions and pressing their thumb against the thumb grip portion.

Preferably, said stem is curved in a plane parallel to the medial plane defined between the mandible and maxilla contacting surfaces of the tooth propping portion. This allows the manually grippable portion to be offset to one side of the mouth of the patient thus allowing good access to the mouth region.

Preferably, the item includes a concave cut-away or scalloped region on the side which faces inwardly in use, to provide increased access for airway tubes etc.

The tooth propping portion is preferably generally H-shaped in cross section comprising a central spacing web and side flanges together defining opposed respective U-shaped recesses for receiving the tooth or gum regions of the mandible or maxilla respectively. Preferably the material of which at least the mandible and maxilla contacting portions is made is soft, with flanges atraumatic on the gums, while being of sufficient extent to hold device in place when patient is unconscious and lying on their side.

In many operations it is required to monitoring end tidal CO₂ to assess the state of the patient. Likewise in other procedures it may be useful to introduce gas into the mouth/throat region of the patient. For these purposes the tooth propping portion and/or the stem may include one or more ports for the passage of gas to or from the interior of the mouth, and a passage connecting said port or ports to a point up the stem from the tooth propping portion. The passageway may be internal and terminate in a connection region for connection in use to a flexible tubular element. To keep the tubular element clear in use, the prop preferably includes a clip region into which said flexible tubular element may be clipped. The clip region may be disposed on the manually grippable portion, for example intermediate the thumb grip portion and one of the cross guard portions. To allow the mouth prop to be used on either side of the mouth, the prop preferably includes two clipping regions provided symmetrically from said manually grippable portion relative to the longitudinal axis of the stem. This also provides the anaesthetist with the option of whether tube clips up or down on either side.

Since it is known that patients' breathe both nasally and orally, it is advantageous for a mouth prop to dispense gases to be breathed both nasally and orally. It is therefore advantageous for the mouth prop to be adapted so that gases to be breathed exit therefrom in the mouth and nasal regions. Additionally, or alternatively, because it is advisable for the wellbeing of the patient to be monitored during anaesthesia or sedation, it is also advantageous for a mouth prop to be adapted to sample exhaled CO₂ and since patients' breathe both nasally and orally it is advantageous for a mouth prop to sample exhaled CO₂ from either or, advantageously both, the mouth and nasal regions. In a preferred embodiment of the invention the mouth prop therefore comprises passage means suitable for delivering gas to be breathed either nasally or orally, or both. Additionally, or alternatively, the mouth prop of the invention is further adapted so as to provide means suitable for sampling exhaled CO₂ from either the nasal or oral region, or both.

It therefore follows that the mouth prop of the invention is relevant during any clinical procedure where the wellbeing of the patient needs to be monitored, typically, by an anaesthetist.

In an alternative embodiment of the invention, a passageway may be provided which passes through a connection region for the connection in use to a tubular element which, in turn, is flexibly connected to a dispensing/collecting means provided with portals for dispensing/collecting selected gases. For example, portals may be fashioned as nasal plugs to either dispense oxygen or collect CO₂. Further, the flexible connection enables the collecting/dispensing means to be positioned in use underneath the patient's nose. Advantageously, in this arrangement the connection region for connection to a tubular element is pivotal so that, depending on whether the mouth prop is to be used on the left or right hand side of the mouth, the tubular element can be swung and ideally located in a fixed position on one or other side of the mouth prop and the dispensing/collecting means can be similarly flexed into the correct position.

Where different gases are required to be delivered to or sampled from the mouth region, the tooth propping and/or the stem may include a further set of one or more ports and an associated connecting passage.

Due to variations in physiology, the required spacing between the maxilla and mandible to achieve the separation between the patient's incisors will vary from patient to patient. Preferably said tooth propping region includes adjustment means to vary the distance between the tooth or gum contacting regions. The adjustment means may include a wedge element movable generally longitudinally between internal contacting surfaces associated with the mandible and maxilla contacting regions. The adjustment means may include a threaded element threadedly engaged at one end with said wedge element and carrying an externally accessible twist handle at the other. Alternatively the adjustment means may include a tie element connected at one end to said wedge element and carrying at its other end an externally accessible actuator element for being moved longitudinally to move said wedge element.

Alternatively, the adjustment means may comprise an internal inflatable space and a passage for supplying and withdrawing inflation fluid to and from said space, whereby the spacing between the maxilla and mandible contacting regions may be varied by introducing or withdrawing fluid from said inflation space.

In another aspect, this invention provides a mouth prop comprising a body portion including a stem at opposite ends of which are provided a tooth propping portion and a manually grippable portion, said tooth propping portion being formed such that it may be inserted in use between the opposed tooth or gum regions of a patient's mandible or maxilla to space the patient's incisors, the tooth propping region and/or the stem including one or more externally open ports for the passing of gas to or from the interior of the mouth, and a passage connecting said port or ports to a point up the stem from the tooth-propping portion.

In another aspect, this invention provides a mouth prop comprising a body portion including a stem at opposite ends of which are provided a tooth-propping portion and a manually grippable portion, said tooth-propping portion being formed such that it may be inserted in use between the opposed tooth or gum regions of a patient's mandible or maxilla to space the patient's incisors, the tooth-propping portion including adjustment means to vary the distance between the regions which contact in use the tooth or gum regions of the mandible and maxilla respectively.

In either of the two aforementioned other aspects of the invention said mouth prop is ideally provided with dispensing and/or collecting means for dispensing gas to be breathed in the mouth and/or nasal region of a patient, or sampling exhaled CO₂ from the mouth and/or nasal region of the patient, respectively.

Whilst the invention has been described above, it extends to any inventive combination of the features set out above or in the following description.

The invention may be performed in various ways, and by way of example only, an embodiment of a mouth prop in accordance with this invention and certain variations will now be described in detail, reference being made to the accompanying drawings, in which:

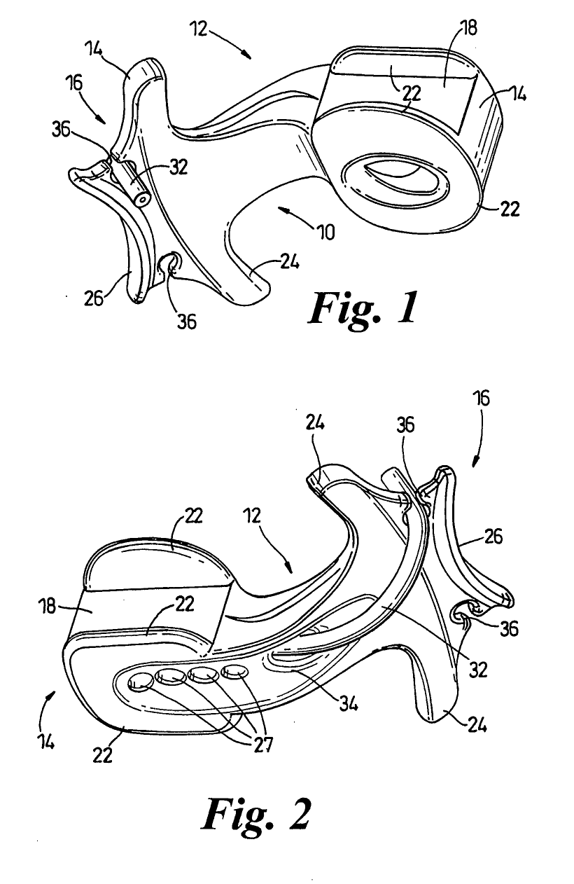

FIG. 1 is a general perspective view of a mouth prop in accordance with this invention, viewed from above and on the outer side;

FIG. 2 is a general perspective view of the mouth prop of FIG. 1, viewed on the inward side;

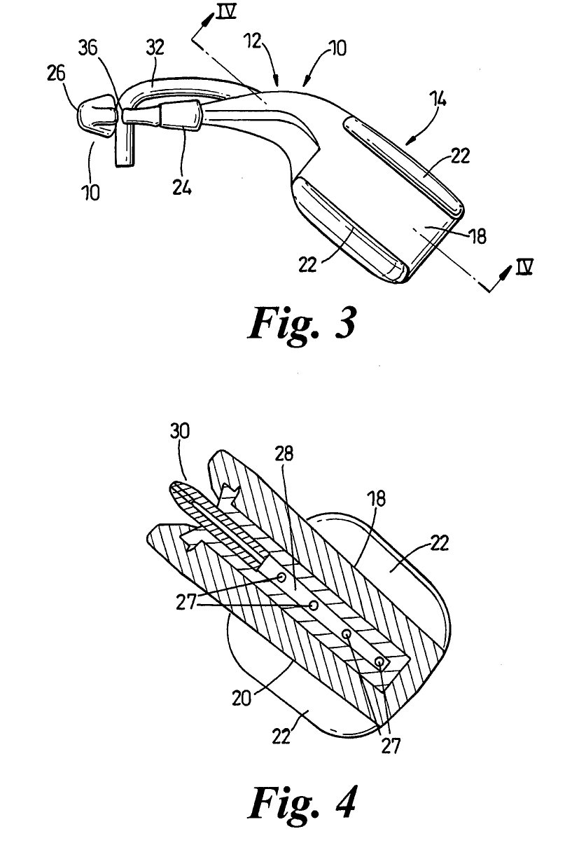

FIG. 3 is a top plan view of the mouth prop of FIG. 1;

FIG. 4 is a section view on lines IV-IV of FIG. 3;

FIG. 5 is an end view of the mouth prop of FIG. 1;

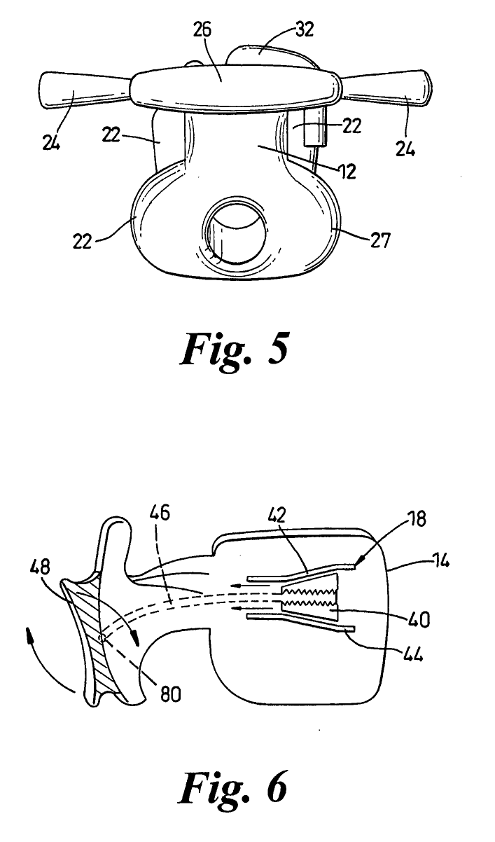

FIG. 6 is a generally schematic view of a first variant design to allow adjustment of the spacing of the mandible and maxilla regions;

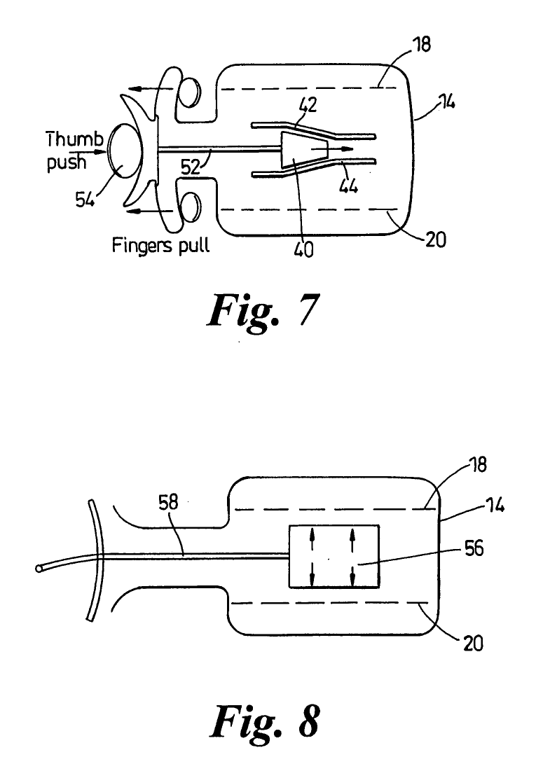

FIG. 7 is a schematic view of a second variation design to allow adjustment of the mandible and maxilla regions;

FIG. 8 is a third variation designed to allow adjustment of the mandible and maxilla regions;

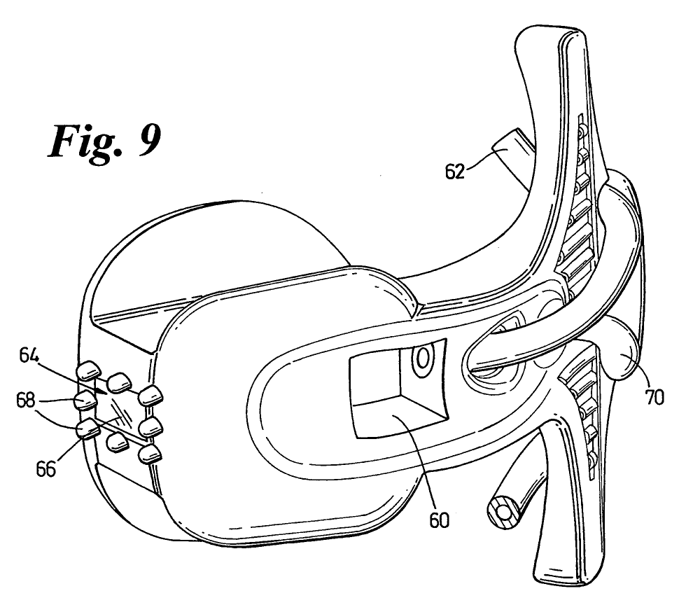

FIG. 9 is a perspective side view of a fourth variation of mouth prop;

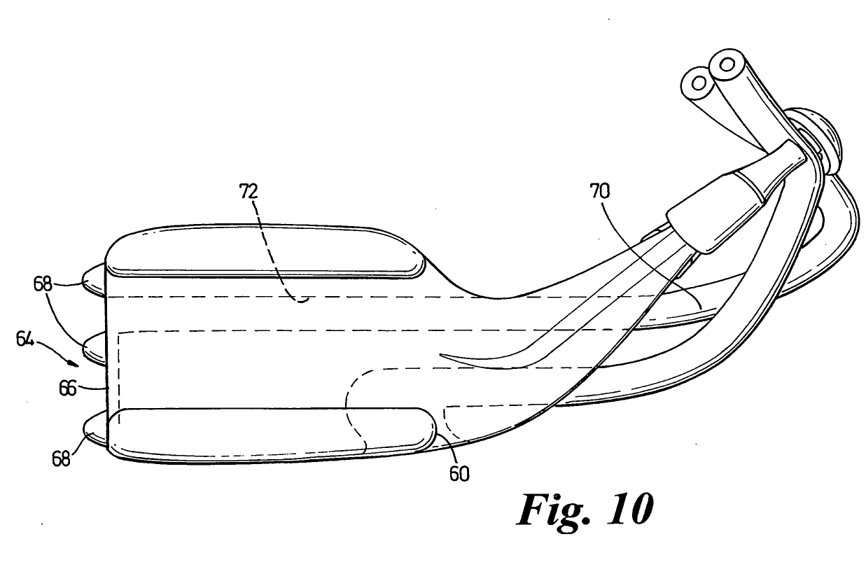

FIG. 10 is a top plan view of the fourth variation of mouth prop;

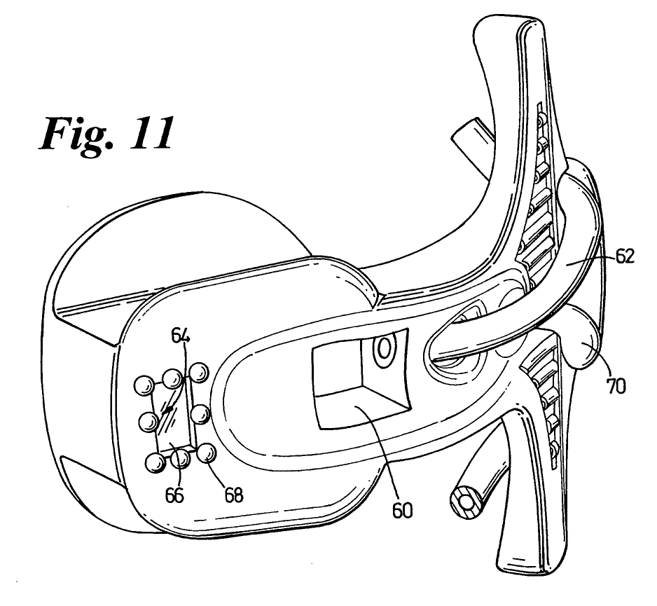

FIG. 11 is a perspective side view of a fifth variation of mouth prop;

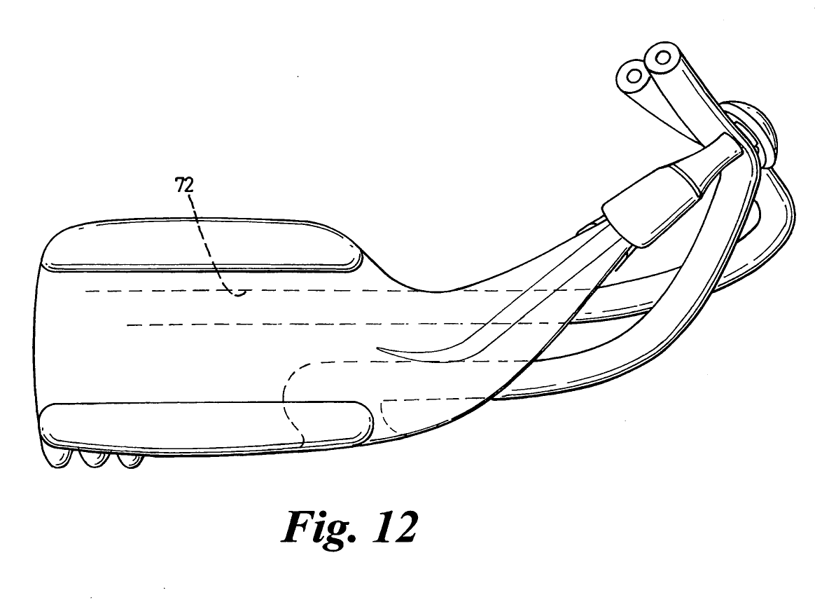

FIG. 12 is a top plan view of the fifth variation of mouth prop;

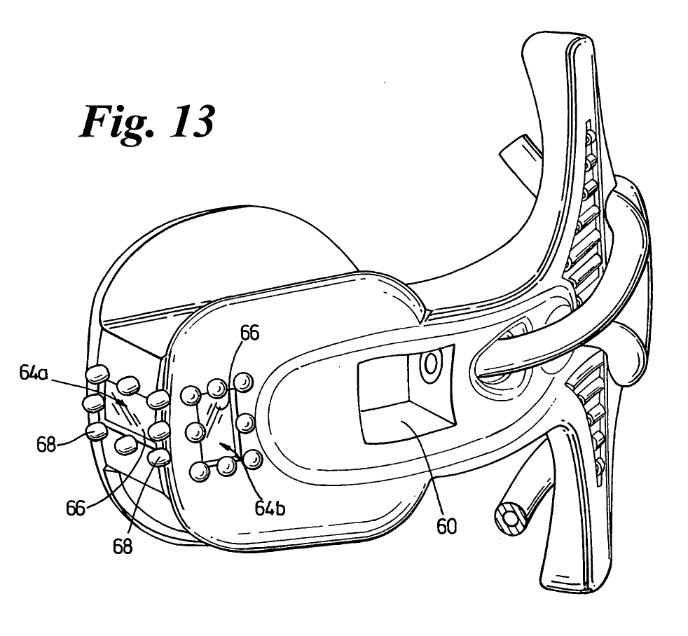

FIG. 13 is a perspective side view of the sixth variation of mouth prop;

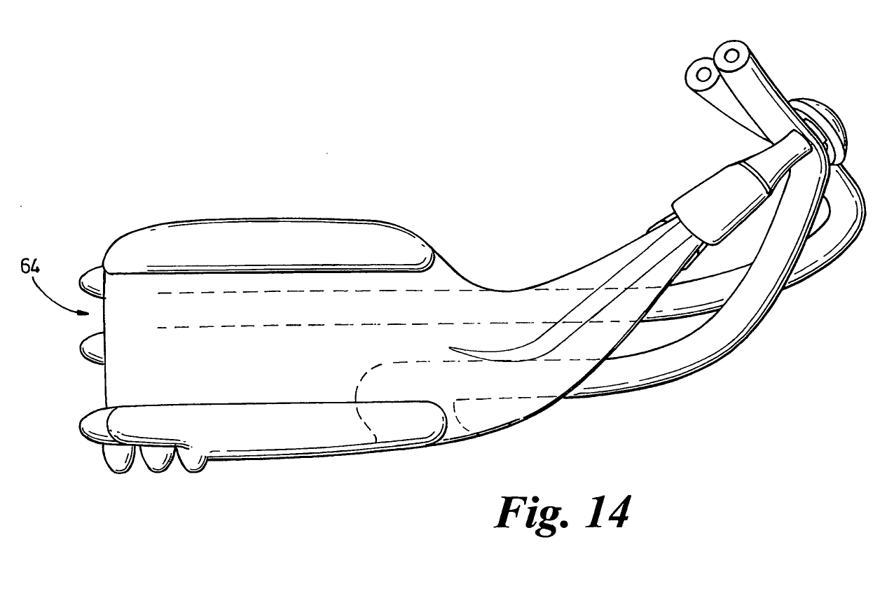

FIG. 14 is a top plan view of the sixth variation of mouth prop;

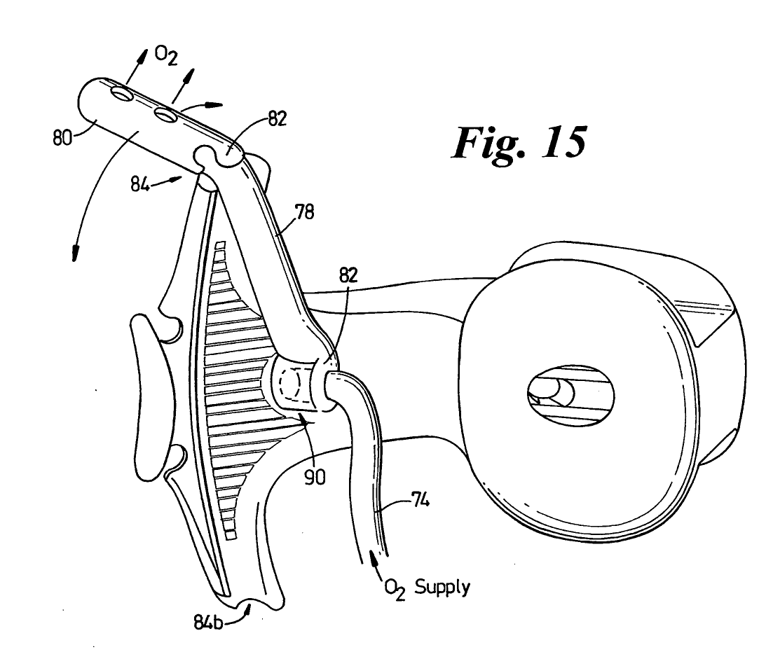

FIG. 15 is a perspective side view of a seventh variation of mouth prop;

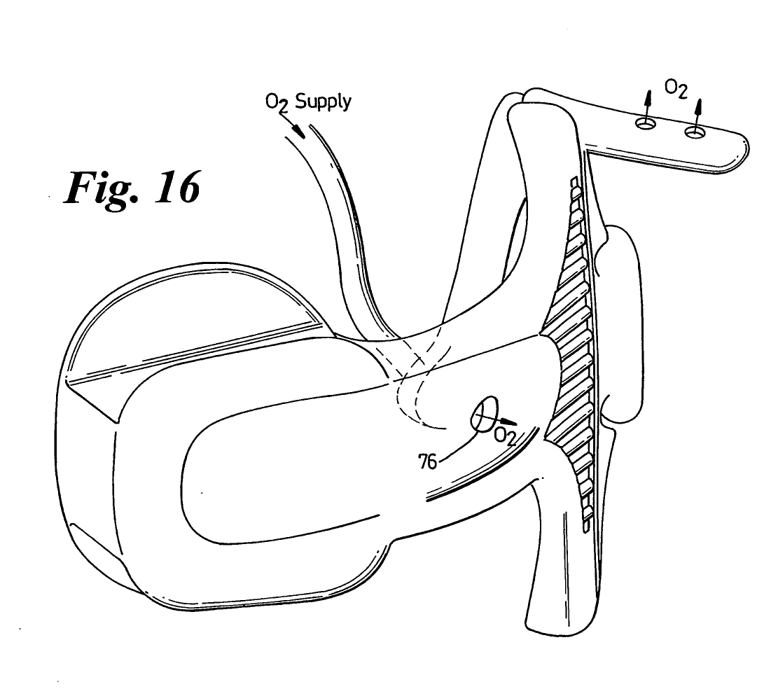

FIG. 16 is a perspective opposite side view of the seventh variation of mouth prop;

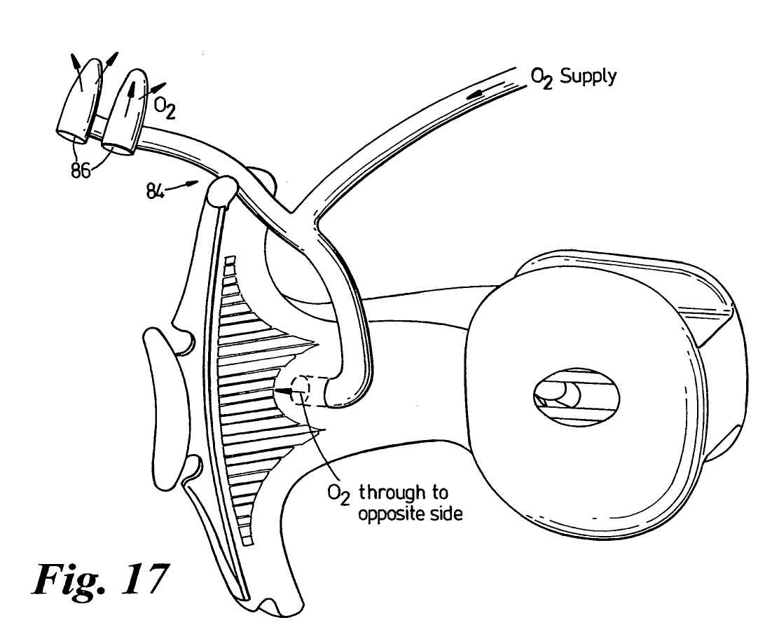

FIG. 17 is a perspective side view of an eighth variation of mouth prop;

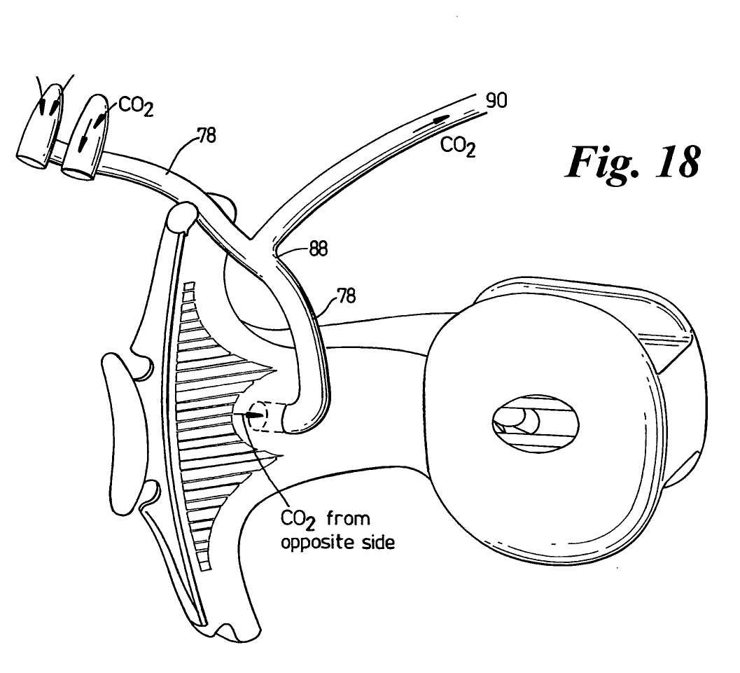

FIG. 18 shows the mouth prop of FIG. 17 when used to sample CO₂;

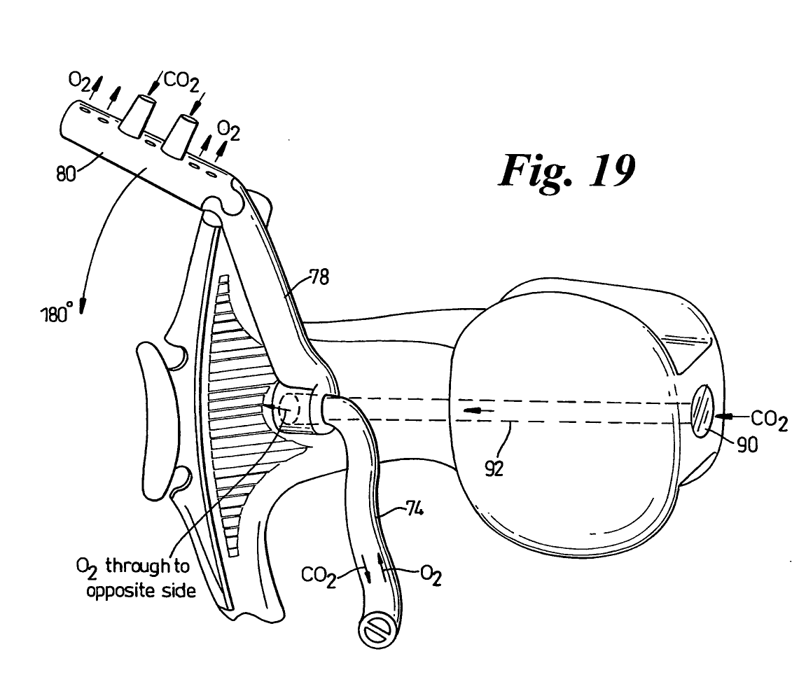

FIG. 19 shows a perspective side view of a ninth variation of mouth prop, and

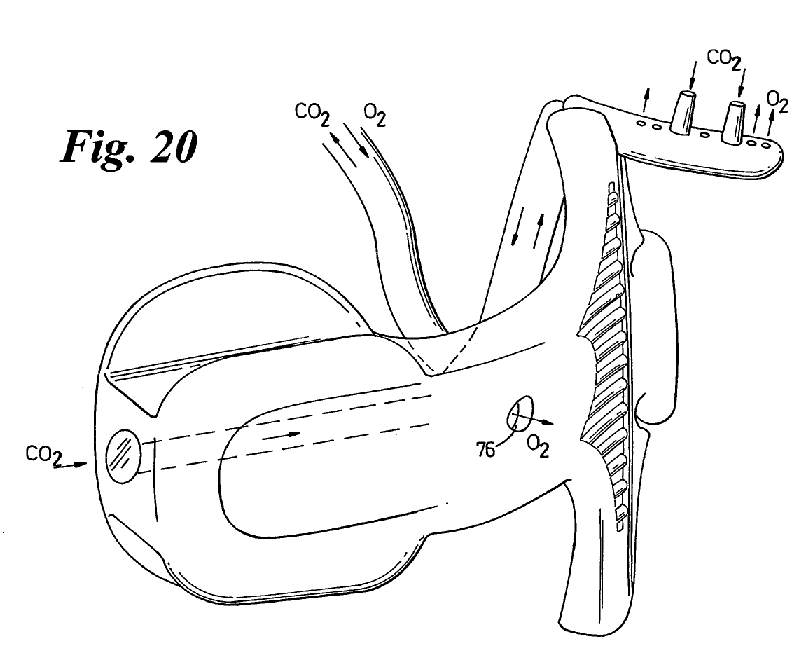

FIG. 20 is a perspective opposite side view of the ninth variation of mouth prop.

Referring initially to FIGS. 1 to 5, the mouth prop 10 of this invention comprises a curved central stem 12, a tooth propping region 14 and a manually grippable portion 16. The tooth propping portion 14 has an upper, maxilla contacting region 18 and a lower mandible contacting region 20 with lateral locating flanges 22 to provide a generally H-sectioned region wherein the flanges provide lateral location for the tooth and/or gum regions of the patient's maxilla and mandible. The stem 12 is curved so that, when the mouth prop 10 is in position between a patient's teeth, the manually grippable portion 16 is located to one side of the mouth, out of the way of the main mouth region and further the mouth prop is fashioned so that only a small portion of the mouth prop handle protrudes from the mouth. Because of this a face mask can be used to ventilate a patient. The manually grippable portion 16 has two lateral cross guard portions 24 and a thumb grip region 26 designed such that the manually grippable portions can be held securely between the thumb and first and second fingers of the clinician, providing stable location. In this embodiment, the tooth propping region 14 is provided on its inner face with a series of four inwardly directed gas ports 27 (see FIGS. 2 and 4) connected to an internal passage 28 (FIG. 4) which terminates in a bored connection spigot 30 onto which may be sealingly push fitted a flexible plastic tube 32. This allows sampling of end-tidal CO₂, or the introduction of oxygen or other gases into the mouth cavity. Although just one set of ports is shown, there may be two sets with respective passages 28 and tubes 32.

From its connection to the spigot 30, the flexible tube 32 passes through a scalloped region 34 on the inner surface of the stem to be secured in a tube clip 36 arranged between the thumb grip portion 26 and the cross guard 24. Another cable clip region 36 is disposed symmetrically on the other side of the prop.

In use, the mouth prop 10 is located between the rear molars of the patient such as to cause the patient's incisors to be spaced by a suitable distance (typically 16-22 mm or more). In this position the stem 12 and manually grippable portion 16 arch laterally to the side of the mouth. It will be noted that the mouth prop is symmetric and so can be used on the left or right hand side of the mouth.

Referring now to the arrangements of FIGS. 6 to 8, in some circumstances it may be required to provide adjustment of the spacing between the maxilla contacting region 18 and the mandible contacting region 20 and the arrangements shown in FIGS. 6 to 8 illustrate how this may be achieved. In each of the arrangements, the tooth propping portion 14 is made of a resiliently deformable material such as to allow the spacing between the surfaces 18 and 20 to be adjusted elastically.

In the arrangement of FIG. 6, an internal wedge 40 is disposed within the tooth propping region 14, co-operating with upper and lower cam surfaces 42,44 such that linear movement of the wedge expands, or allows elastic retraction, of the tooth gripping portion 14. In FIG. 6 the wedge 40 is provided with an internal threaded bore which engages the threaded end of a flexible shaft 46 which extends through the stem and is attached to an external rotatable thumb twist handle 48 by a pivot 50. In this arrangement the spacing between the upper and lower regions 18 and 20 is done by twisting the thumb twist handle 48 to move the wedge 40 back and forth.

Referring now to the embodiment of FIG. 7, in this arrangement there is a wedge 40 again disposed between upper and lower cam surfaces 42 and 44 and in this arrangement the wedge may be pushed linearly to expand the tooth propping portion to increase the distance between the surfaces 18 and 20. For this purpose the wedge is connected via a flexible push rod 52 to a thumb loop 54. Suitable locking or ratchet mechanisms may be provided.

In the embodiment of FIG. 8, the tooth propping portion 14 is provided with an internal inflation space 56 which is connected via a passage 58 to an external output port 60. The spacing between the upper and lower surfaces is achieved by introducing or withdrawing air or other inflation medium to the space 56 through the passage 58. This could be done typically by a syringe or hand pump which may be integral to the device or separate.

Referring now to the variants shown in FIGS. 9 to 14, these each show arrangements of mouth prop where, as set out earlier in the specification, the mouth prop may be used both to deliver oxygen and to sample CO₂. Thus, in the embodiments of FIGS. 9 and 10, a recess 60 for connection to an oxygen delivery pipe 62 is disposed on the inner face of the mouth prop so as to discharge oxygen generally inwardly. At the end of the device is provided a CO₂ monitoring port 64 which is covered by a hydrophobic filter 66 to prevent the ingress of saliva/blood/body fluid which would otherwise cause cross-contamination of the sampling lines and monitoring equipment. The CO₂ monitoring port is directed along the axis of the device whilst the oxygen recess 60 is designed to discharge oxygen generally transversely, to reduce the possibility of cross-contamination with the CO₂ sampling and also oxygen directed to the back of the throat can cause a gag reflex. Furthermore, the recess 60 is of relatively large size so that it prevents ‘jetting’ of oxygen. The CO₂ monitoring port 64 is surrounded by a series of dimples 68 which prevent the tongue or the mucosal membranes from blocking the sampling port 64. As can be seen more clearly in FIG. 10, a CO₂ tube 70 passes along a bore 72 to terminate adjacent the CO₂ monitoring port 64. Again, as may be seen from FIG. 10, the oxygen recess 60 may be curved at the edges to promote the required distribution of O₂.

Referring now to the embodiment of FIGS. 11 and 12, here the oxygen delivery port 60 is of similar form to that of the embodiment of FIGS. 9 and 10. The difference in this embodiment is that the CO₂ monitoring port 64 is aligned transversely to the body of the prop and so its sampling direction is generally parallel to but spaced as far as possible from, the delivery direction of the O₂ port 60.

In FIGS. 13 and 14, the two previous arrangements are combined so that there is a single O₂ delivery port 60 but two CO₂ monitoring ports 64a and 64b, with one disposed perpendicularly to the O₂ delivery direction and the other disposed generally parallel. In this arrangement, should one of the CO₂ ports become obstructed, sampling will still be possible from the other port.

In FIGS. 15 and 16 an alternative embodiment of a mouth prop is shown and moreover a functionality is illustrated i.e. supplying a patient with oxygen to breathe. Oxygen is supplied via supply 74 to a bifurcating junction 90 whereat oxygen can exit the mouth prop via hole 76 (best seen in FIG. 16) located just inside a patient's incisors, and so blowing oxygen into the open space at the front of the mouth, or travel via tubular element 78 to a dispensing means 80. The upper end of tubular element 78 rests stably in clip 84. Further, tubular element 78 is flexibly connected to dispensing means 80 via flexible joint 82. Additionally, tubular element 78 is rotatably mounted with respect to the mouth prop so that in use tubular element 78 can be disengaged from its resting position on clip 84 and swung round to lower clip 84b. Once in place on lower clip 84b the dispensing means 80 can be repositioned, via flexible joint 82, under a patient's nose. In this way, the device can be used on either the right or left hand side of the mouth in order to deliver gas to be breathed, in this instance oxygen, to the buccal cavity and the nose. However, the apparatus can be used in a reverse functionality to extract exhaled CO₂ from both the mouth and the nose by simply applying an appropriate negative pressure.

In FIGS. 17 and 18 there is shown an alternative embodiment of a mouth prop. In FIG. 17 the apparatus for dispensing gas to be breathed comprises tubing that can maintain its position once deformed in a certain manner. Thus, ideally, the tubing is reinforced with a suitable stiffener so that it can be bent around the mouth prop and, in a first orientation, made to rest on upper latch 84 and then further deformed so that the gas dispensing means, in this instance, nasal plugs 86, are positioned inside the nasal orifices. Although not shown, the opposite side of this embodiment of the invention comprises orifice 76 through which gas is dispensed to the mouth.

As an alternative embodiment the dispensing means is provided with any other means suitable for stably positioning the oxygen dispensing means, such as the nasal plugs, in or adjacent the nasal orifices. For example, the tubing may be suitably concertinaed and stiffened so as to provide it with sufficient support to maintain the orientation of the gas dispensing means with respect to a patient's nose.

In FIG. 18 the device of FIG. 17 is shown when used for sampling exhaled CO₂. In this embodiment suitable pressure is applied in order to draw CO₂ from both the nasal cavity and, via hole 76, the mouth. CO₂ travels along collecting tube 78 towards junction 88 where nasally and orally exhaled CO₂ mix and ultimately travel along sampling tube 90 towards a suitable sampling means. Ideally, junction 88 is positioned so that the distance travelled by exhaled CO₂ from the mouth is equal to the distance travelled by exhaled CO₂ from the nose. This embodiment is preferred because it overcomes irregularities in sampling techniques.

In FIG. 19 there is shown yet a further mouth prop. In this embodiment the mouth prop is adapted to not only dispense gas to be breathed but also to sample exhaled CO₂. It therefore contains a number of tubes or passageways for delivering gas to be breathed to both the mouth and the nose and sampling exhaled CO₂ from both the mouth and the nose. The configuration is very similar to that described in relation to FIGS. 15-18 but within supply tube 74 there is provided a separate passageway for extracting CO₂ from a patient using the device. Similarly, within tube 78 there is provided, not only a passageway for delivering oxygen to dispensing means 80, but also a passageway or tube for extracting exhaled CO₂ from the nasal region of a patient. As seen in FIG. 20, the opposite side of the mouth prop comprises hole 76 through which oxygen is dispensed to the mouth region of the patient.

To facilitate the collection of exhaled CO₂ from the mouth a rearward cavity 90 is provided. Cavity 90 is in fluid communication with an internal passageway 92 in the mouth prop. Further passageway 92 leads into the CO₂ passage in tube 74. It therefore follows that exhaled CO₂ in the mouth is extracted from the throat region of an individual. In the nasal region nasal prongs are provided for the extraction of CO₂ and so penetrate the nasal orifices. In this way extraction of CO₂ occurs within the patient and, in any event, rearward of the delivery of gases to be breathed. This is an advantageous way of sampling exhaled CO₂.

The invention therefore concerns the provision of a novel mouth prop that can be adapted to deliver gases to be breathed and/or sample exhaled gases.

1. A mouth prop comprising a body portion including a stem at opposite ends of which are respectively provided a tooth propping portion and a manually grippable portion, said tooth propping portion being formed such that it may be inserted in use between the opposed tooth or gum regions of a patient's mandible or maxilla to space the patient's incisors, the manually grippable portion having transversely arranged cross guard portions around which in use a clinician may hook their first and second fingers, for assisting location and removal of the mouth prop.

2. A mouth prop according to claim 1, wherein said cross guard portions are sized to prevent swallowing of the mouth prop.

3. A mouth prop according to claim 1, wherein said manually grippable portion further includes a thumb grip portion disposed to allow a clinician in use to hold the prop securely by hooking their first and second fingers around the cross guard portions and pressing their thumb against the thumb grip portion.

4. A mouth prop according to claim 1, wherein said stem is curved in a plane parallel to the medial plane defined between the mandible and maxilla contacting surfaces of the tooth propping portion.

5. A mouth prop according to claim 4, wherein said stem includes a concave cutaway or scalloped region on the side which faces inwardly in use, thereby to provide increased access for, or to act as a guide for, airway tubes of the cuffs thereof or scopes.

6. A mouth prop according to claim 1, wherein said tooth propping portion is generally H-shaped in cross section, comprising a central spacing web and side flanges together defining opposed respective U-shaped recesses for receiving the tooth or gum regions of the mandible or maxilla respectively.

7. A mouth prop according to claim 1, wherein the tooth propping region and/or the stem includes one or more ports for the passage of gas to or from the interior of the mouth, and a passage connecting said ports to a point up the stem from the tooth propping portion.

8. A mouth according to claim 7, wherein said passage is internal and terminates in a connection region for connection in use to a flexible tubular element.

9. A mouth prop according to claim 8, wherein said prop includes at least one clip region into which said flexible tubular element may be clipped in use.

10. A mouth prop according to claim 9, wherein said clip region is disposed on the manually grippable portion.

11. A mouth prop according to claim 10, wherein two clipping regions are symmetrically disposed on said manually grippable portion relative to the longitudinal axis of the stem.

12. A mouth prop according to claim 7, wherein the tooth propping region and/or the stem include a further set of one or more ports, and a connecting passage.

13. A mouth prop according to claim 1, wherein said tooth propping region includes adjustment means to vary the distance between the regions which, in use, contact the tooth or gum regions of the mandible and maxilla respectively.

14. A mouth prop according to claim 13, wherein said adjustment means includes a wedge element moveable generally longitudinally between internal contacting surfaces associated with the mandible contacting region and the maxilla contacting region respectively.

15. A mouth prop according to claim 14, wherein said adjustment means includes a threaded element, threadedly engaged at one end with said wedge element and carrying an externally accessible twist handle at the other.

16. A mouth prop according to claim 14, wherein said adjustment means includes a tie element connected at one end to said wedge element and carrying at its other end an externally accessible actuating element for being moved to move said wedge element.

17. A mouth prop according to claim 13, wherein said adjustment means comprises an internal inflatable space and a passage for supplying and withdrawing inflation fluid to and from said space, whereby the spacing between the maxilla and mandible contacting regions may be varied by introducing or withdrawing fluid.

18. A mouth prop comprising a body portion including a stem at opposite ends of which are provided a tooth propping portion and a manually grippable portion, said tooth propping portion being formed such that it may be inserted in use between the opposed tooth or gum regions of a patient's mandible or maxilla to space the patient's incisors, the tooth propping region and/or the stem including one or more externally open ports for the passing of gas to or from the interior of the mouth, and, optionally, a passage connecting said port or ports to a point up the stem from the tooth-propping portion.

19. A mouth prop comprising a body portion including a stem at opposite ends of which are provided a tooth-propping portion and a manually grippable portion, said tooth-propping portion being formed such that it may be inserted in use between the opposed tooth or gum regions of a patient's mandible or maxilla to space the patient's incisors, the tooth-propping portion including adjustment means to vary the distance between the regions which contact in use the tooth or gum regions of the mandible and maxilla respectively.

20. A mouth prop according to claim 18, wherein said one or more ports comprise a forward port which discharges generally transversely to the axis of the body portion.

21. A mouth prop according to claim 20, wherein said port includes a recess with which gas may discharge and thence pass in use within the user's mouth.

22. A mouth prop according to claim 18, wherein said one or more ports comprise at least one rearward sampling port.

23. A mouth prop according to claim 22, wherein said sampling port samples in a direction parallel to the axis of said body portion.

24. A mouth prop according to claim 22, wherein said sampling port samples in a direction generally perpendicular to the axis of the body portion.

25. A mouth prop according to claim 22 wherein there are two sampling ports.

26. A mouth prop according to claim 22, wherein the or each said sampling port has a filter element.

27. A mouth prop according to claim 22, wherein one or more projecting elements are disposed around the periphery of the aperture to prevent blocking.

28. A mouth prop according to claim 18, wherein one of said ports terminates in a connection region for connection in use to a tubular element.

29. A mouth prop according to claim 28, wherein said tubular element is flexible.

30. A mouth prop according to claim 28, wherein said tubular element is rigid or semi-rigid.

31. A mouth prop according to claim 29, wherein said tubular element terminates in a dispensing and/or collecting means.

32. A mouth prop according to claim 31, wherein the dispensing and/or collecting means is flexibly or pivotally connected to the tubular element.

33. A mouth prop according to claim 28, wherein said tubular element is rotatably mounted on said mouth prop.

34. A mouth prop according to claim 28, wherein said tubular element comprises a plurality of passageways wherein selected passageways are connected to selected ports/portals.

35. (canceled)
